(19) United States
(12) Patent Application Publication
(10) Pub. No.: US 2006/0167522 A1
(43) Pub. Date: Jul. 27, 2006

(54) Connector for use in an implantable stimulator device

(76) Inventor: Zdzislaw B. Malinowski, Castaic, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/043,640

(22) Filed: Jan. 25, 2005

(51) Int. Cl. A61N 1/375

(52) U.S. Cl. 607/37

(57) ABSTRACT

A connector is configured to couple an implantable pulse generator (IPG) to an electrical stimulation lead or electrical leads while allowing the implantable pulse generator to be hermetically sealed within a case assembly. The connector includes a resilient body having a lead insertion lumen defined therein. Connector contacts for connecting to multiple contacts at the proximal end of the stimulation may be disposed along the length of the insertion lumen as an array. The connector contacts are configured to be coupled to lead extensions or leads, which direct electrical stimuli to a desired body location.

### BACKGROUND

Spinal cord stimulation systems and other stimulation devices frequently include an implantable pulse generating system for treating chronic pain by providing electrical stimulation pulses from an electrode array placed epidurally near a patient's spine. Spinal cord stimulation (SCS) is a well-accepted clinical method for reducing pain in certain populations of patients. SCS systems typically include an implanted pulse generator (IPG), a stimulation lead, and electrode contacts connected to the distal portion of the stimulation lead. Traditional SCS systems may also include a lead extension placed between the IPG and the stimulation lead.

The pulse generator generates electrical pulses that are delivered to the dorsal column fibers within the spinal cord through the electrodes, which are implanted along the dura of the spinal cord. In a typical situation, the attached lead wires exit the spinal cord and are tunneled around the torso of the patient to a sub-cutaneous pocket where the pulse generator is implanted.

In order to protect the electronic circuitry of the pulse generator from environmental conditions and/or other damage while the IPG is implanted within a patient, the IPG is frequently enclosed in a titanium case. The titanium case is configured to provide protection and a hermetic, or completely sealed, environment. For example, the titanium case frequently includes two halves. Recesses are formed in each of the halves such that when the two halves are coupled together, holes are defined therein. A feedthru member extends through the defined holes to allow the lead wires to be electrically coupled to the electronic circuitry of the IPG while maintaining the hermeticity of the titanium case.

Traditionally, the interconnection between an IPG or other neurostimulator device and the stimulating leads is formed with a hard epoxy header. The header includes at least one fixed lead insertion hole which accepts the proximal connector portion of a stimulating lead. Inside the header, some type of mechanical connection is provided to connect each of the multiple contacts on the proximal connector portion of a multi-contact lead to the electronic circuitry of the IPG. One such mechanical connection is a bal seal (Bal Seal Engineering Company, Foothill Ranch, Calif.). A bal seal provides physical and electrical connection to the multiple contacts on the lead connector through a compressive contact. In addition, in order to ensure that the lead connector is securely locked into the IPG header and cannot slip out, a set screw is often employed to compress a portion of the stimulating lead connector to thereby positively lock the stimulating lead into the lead insertion hole.

Disadvantageously, the use of a large setscrew to compress the lead connectors at the proximal connector end can create internal stresses on the feedthru pins and the hard epoxy comprising the header. Consequently, the material in the feedthru member construction, which must ensure hermeticity, is under constant stress and may develop cracks and eventually permit a leak into the stimulator electronics. This mechanism of failure may result in corrosion and eventual malfunction of the stimulator device, which in turn will result in having to explant the device. Further, the setscrew traditionally used for locking stimulating leads within the body of the header may cause lead distortion which may make it difficult to remove the lead connector from the header at a future date. More specifically, using a set screw allows the clinician to excessively tighten the setscrew as precise torque applied to the setscrew is at the discretion of the clinician. Excessive tightening can damage the IPG header and the lead connector, which damage, if identified, results in scrapping both the lead and IPG. If the damage is not identified, post-implant leakage of the header and intermittent connections between the lead connector and feedthru contacts may occur. Moreover, traditional IPG headers are permanently attached to the IPG case and have a fixed lead insertion hole size, thereby limiting the stimulating lead connector size that may be received therein.

### SUMMARY

An embodiment of a connector is provided herein for use in a stimulator device. In particular, the connector is configured to provide zero insertion force coupling of an implantable pulse generator to electrical leads while allowing the implantable pulse generator to be hermetically sealed within a case assembly. For example, according to one exemplary embodiment, the connector includes a resilient body having a lumen defined therein. Connector block contacts are disposed along the length of the lumen. The connector contacts are configured to be coupled to lead extensions or leads, which direct electrical stimulation to a desired body location.

In one exemplary embodiment, it is a feature to provide a removable connector for use in a stimulator device that has substantially zero-insertion force;

It is another feature of an exemplary embodiment to optionally provide a connector that does not require a set screw that contacts and secures the end of a stimulating lead;

It is a further feature of one exemplary embodiment to provide a connector block that is relatively clear so that the male end of an extension lead or the proximal connector end of a stimulating lead can be seen as it is inserted into the insertion lumen or lumens within the connector block to facilitate a correct insertion;

It is yet a further feature of an exemplary embodiment to permit easy replacement of a removable connector block having a differently configured and dimensioned insertion lumen to accept a lead extension or stimulation lead with a differently sized and configured proximal connector end.

One exemplary connector operates by applying compressive force on or around the connector block which permits compression. Compression of the connector block is then transferred to the connector contacts on the connector of the proximal end of a lead, thereby locking the lead in the connector block.

### BRIEF DESCRIPTION OF THE DRAWINGS

The accompanying drawings illustrate various embodiments of the present apparatus and method and are a part of the specification. The illustrated embodiments are merely examples of the present apparatus and method and do not limit the scope of the disclosure.

FIG. 1 illustrates an exploded perspective view of a stimulator device that includes a zero insertion force resilient connector, according to one exemplary embodiment.

FIG. 2 illustrates an exploded perspective view of a zero insertion force connector and a feedthru member, according to one exemplary embodiment.

FIG. 3A illustrates a perspective view of a case frame, according to one exemplary embodiment.

FIG. 3B illustrates a perspective view of a connector block cover, according to one exemplary embodiment.

FIG. 3C illustrates a perspective view of a removable connector block being inserted into a case frame, according to one exemplary embodiment.

FIG. 3D is a perspective view illustrating a removable connector block being seated in a feedthru opening formed in a case frame, according to one exemplary embodiment.

FIG. 3E is a perspective view illustrating a number of lead extensions or ends of stimulation leads being inserted into a connector block, according to one exemplary embodiment.

FIG. 3F is a perspective view illustrating a plurality of lead extensions or ends of stimulation leads coupled to a stimulator device through a removable connector block, according to one exemplary embodiment.

FIG. 4A is a side view illustrating a number of forces exerted on the connector block by the connector block cover when the connector block cover is locked, according to one exemplary embodiment.

FIG. 4B is a front view illustrating a number of forces exerted on the connector block and the connector contacts when a connector block cover is locked, according to one exemplary embodiment.

FIG. 5 is a perspective view illustrating a connector block cover prying tool, according to one exemplary embodiment.

FIGS. 6A through 6C illustrate perspective views of a prying tool being engaged with and releasing a connector block cover from its locked position, according to one exemplary embodiment.

Throughout the drawings, identical reference numbers designate similar, but not necessarily identical, elements.

### DETAILED DESCRIPTION

A connector is provided herein for use in a stimulator device (e.g., an implantable pulse generator or IPG). In particular, one embodiment of the connector is configured to provide little or no resistance to the insertion of electrical leads while securely coupling an IPG to the electrical leads or lead extensions. For example, according to one exemplary embodiment, the connector includes a connector block comprised of a resilient, compressible body having a lead insertion lumen defined therein. Connector contacts are disposed along the length of the lead insertion lumen. The connector contacts, forming an array, are configured to be coupled to lead extensions or leads that are inserted in the lumen, which lead extensions or leads direct electrical stimulation to a desired location in the body.

According to one exemplary embodiment, when the lead extensions or leads are inserted into the lumen, parts of the lead extensions or leads are also passed through the connector contacts. Once the lead or lead extension is inserted into the lumen, the connector may be subjected to compressive forces that are transferred through the connector to the connector contacts. The compressive forces resiliently clamp the connector contacts to the lead extensions. The connector contacts are also coupled to feedthru pins which in turn are coupled to a feedthru member and the IPG. As a result, the connector block provides an electrical pathway from the IPG to leads or lead extensions. Further details of the exemplary connector and its uses will be given below.

In the following description, for purposes of explanation, numerous specific details are set forth in order to provide a thorough understanding of the present method and apparatus. It will be apparent, however, to one skilled in the art, that the present method and apparatus may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

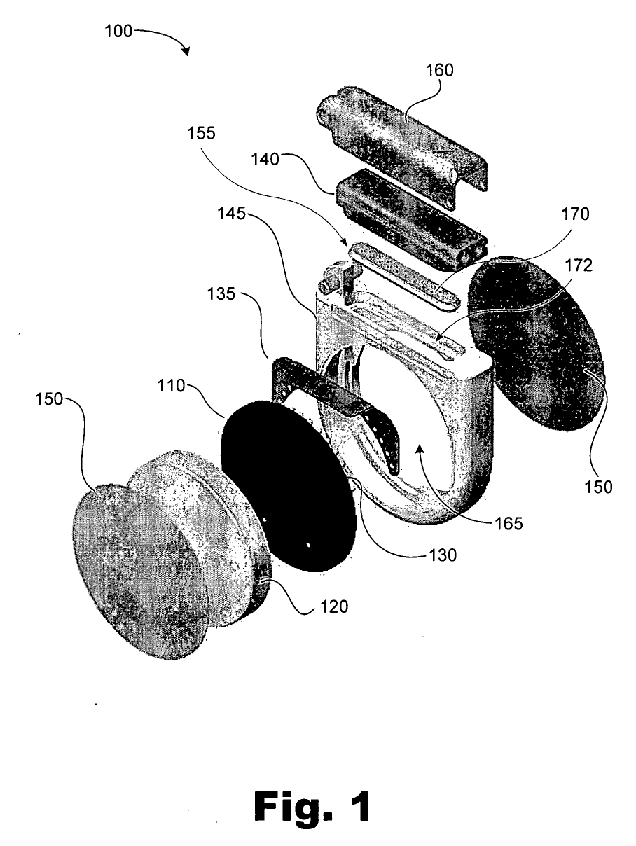

FIG. 1 illustrates an exploded perspective view of an implantable stimulator device or IPG (100) that may include the connector device, in accordance with the present exemplary embodiment. The IPG (100) includes several components coupled to a hybrid circuit board (110), a power source (120), and hybrid pins (130). The circuit board (110) includes electronic circuitry populated thereon. This circuit board (110) draws power from the power source (120) and delivers electrical stimulation energy through the hybrid pins (130). The hybrid pins (130) electrically couple the circuit board (110) to a flexible or “flex” connector (135), which in turn is connected to feedthru pins (170) formed on the feedthru member (155). In particular, each of the hybrid pins (130) is associated with a corresponding opening defined in the flexible connector (135). The flexible connector (135) in turn is coupled to a connector block (140) via the feedthru member (155). The hybrid circuit board (110) and the power source (120) are hermetically sealed within a case assembly that includes a case frame (145), and side lids (150). In particular, the case frame (145) includes a cavity (165) formed therein that is configured to have the circuit board (110) and power source (120) contained therein.

The hybrid circuit board or electronic circuit board (110) and the power source (120) may be coupled together, such as through welding or the application of conductive adhesive. Once the power source (120) is coupled to the circuit board (110), they may be placed within the cavity (165) of the case frame (145) and the side lids (150) may be sealingly coupled to the case frame (145). The side lids (150) may be metallic, e.g., titanium, or non-metallic, e.g., ceramic or a plastic. Accordingly, the side lids (150) may, in some embodiments, e.g., where the side lids are ceramic or metallic, hermetically seal the sides of the case frame (145).

The circuit board (110) is formed by electrically coupling electronic components to the circuit board. According to one exemplary method, the components of the circuit board (110) are physically coupled to the circuit board using solder or conductive epoxy. These components may include, but are in no way limited to, a microcontroller coupled to a memory circuit. An exemplary microcontroller includes a microprocessor and associated logic circuitry, which in combination with control logic circuits, timer logic, and an oscillator and clock circuit, generates the control and status signals that allow the microcontroller to control the operation of the IPG (100) in accordance with a selected operating program and stimulation parameters.

The operating program and stimulation parameters are typically programmably stored within the memory circuitry by transmitting an appropriate modulated carrier signal through a receiving coil and charging and forward telemetry circuitry from an external programming unit, such as a handheld programmer (HHP) and/or a clinician programmer (CP), assisted as desired through the use of a directional device. The handheld programmer may thus be considered to be in “telecommunicative” contact with the IPG. Similarly, the clinician programmer is considered to be in telecommunicative contact with the handheld programmer and, through the handheld programmer, with the IPG. The charging and forward telemetry circuitry demodulates the carrier signal it receives through the coil to recover the programming data (for example, the operating program and/or the stimulation parameters), which programming data is then stored within the memory or within other memory elements distributed throughout the circuit board (110).

The microcontroller is further coupled to monitoring circuits via a bus. The monitoring circuits monitor the status of various nodes or other points throughout the IPG (e.g., power supply voltages, current values, temperature, the impedance of electrodes attached to the various electrodes, and the like). Informational data sensed through the monitoring circuit may be sent to a remote location external to the IPG (e.g., a non-implanted location) through back telemetry circuitry, including a transmission coil.

The circuit board (110) also includes power circuits. The power circuits may include protection circuitry that protects a replenishable power source from overcharging. Further, safeguarding features may be incorporated that help assure that the power source is operated in a safe mode upon approaching a charge depletion. Potentially endangering failure modes are reduced and/or prevented through appropriate logic control that is hard-wired into the device or otherwise set in the device in such a way that a patient cannot override them.

As previously discussed, the circuit board (110) is coupled to the power source (120). According to one exemplary embodiment, the power source (120) may be coupled to the circuit board (110) by soldering or by the use of conductive epoxy. Any other suitable process for coupling the power source (120) to the circuit board (110) may also be used.

The power source (120) may include a primary, non-rechargeable battery, a rechargeable battery, and/or a super-capacitor. Such a power source provides an unregulated voltage to power circuits. The power circuits, in turn, generate the various voltages, some of which are regulated and some of which are not, as needed by the various circuits located within the circuit board (110). The power circuits further selectively direct energy contained within the carrier signal, obtained through the charging and forward telemetry circuit, to the replenishable power source (120) during a charging mode of operation. In this manner, the replenishable power source (120) may be recharged.

According to one exemplary embodiment, the power source (120) includes a rechargeable battery, and more particularly, a rechargeable Lithium Ion battery. The power source (120) may be recharged inductively from an external charging station. Further, an internal battery protection circuitry may be used for safety reasons, such as to prevent the battery from being overcharged and/or to only accept a charge from an authorized charging device.

The case frame (145) also includes a feedthru opening (172) defined therein. The feedthru opening (172) according to the present exemplary embodiment extends through the case frame (145) and into the cavity (165). The removable connector block (140) is configured to be placed at least partially within the feedthru opening (172) to form an electrical connection with the feedthru member (155).

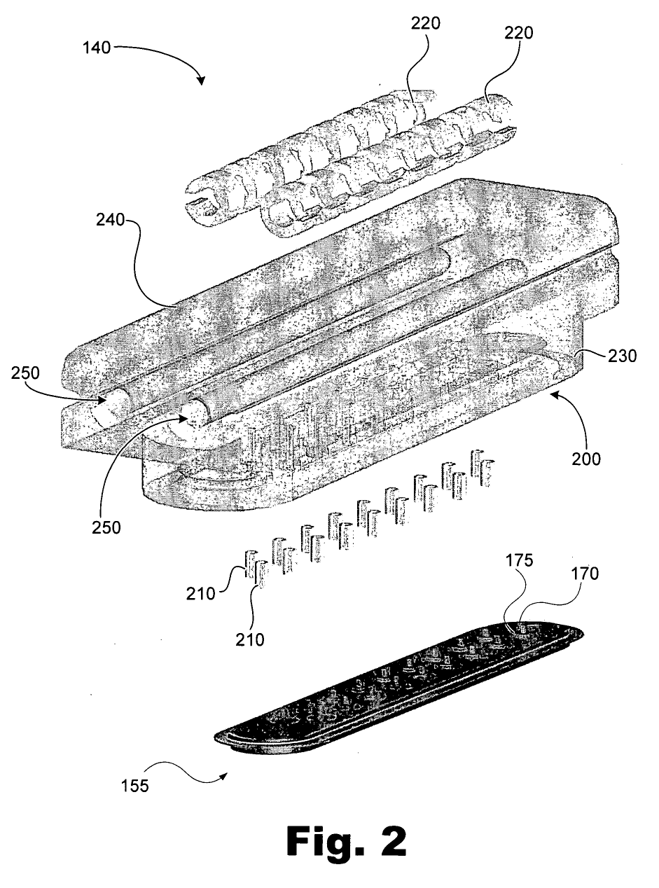

A feedthru member (155) may be secured in the feedthru opening (172) as illustrated in FIG. 1. As shown, the feedthru member (155) includes a number of feedthru pins (170) that provide the electrical connection between the flexible connector (135) hermetically sealed within the case frame (145) and the removable connector block (140). According to one exemplary embodiment, the feedthru member (155) includes a flange that is configured to seat in the feedthru opening (172) and be sealingly coupled to the case frame (145). The feedthru member (155) may be sealingly coupled to the case frame by an adhesive, welding, and the like. The feedthru member (155) provides the electrical connection between the circuit board (110) and the connector block (140) via a number of feedthru pins (170) that extend through both sides of the feedthru member. According to one exemplary embodiment, the feedthru pins (170) are made of a conductive material such as platinum and are surrounded by an insulating material (175; FIG. 2) such as glass to electrically isolate each feedthru pin. When assembled, the feedthru pins (170) electrically couple the flexible connector (135) and the connector block (140).

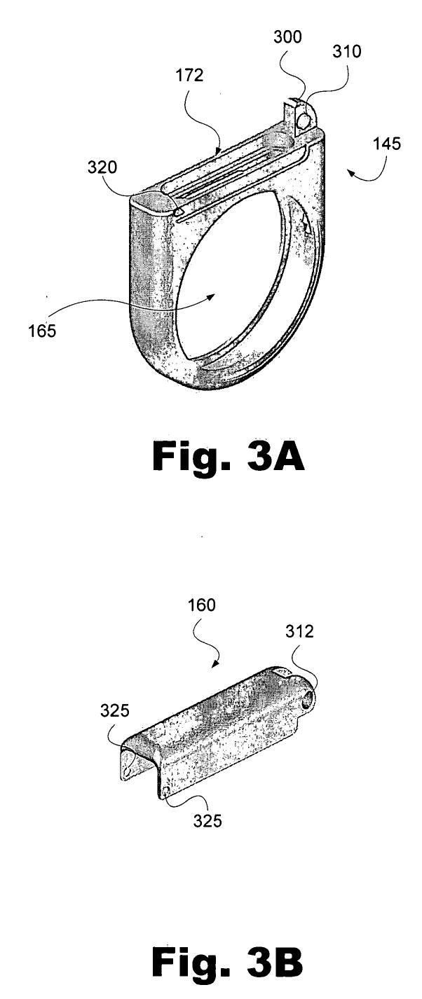

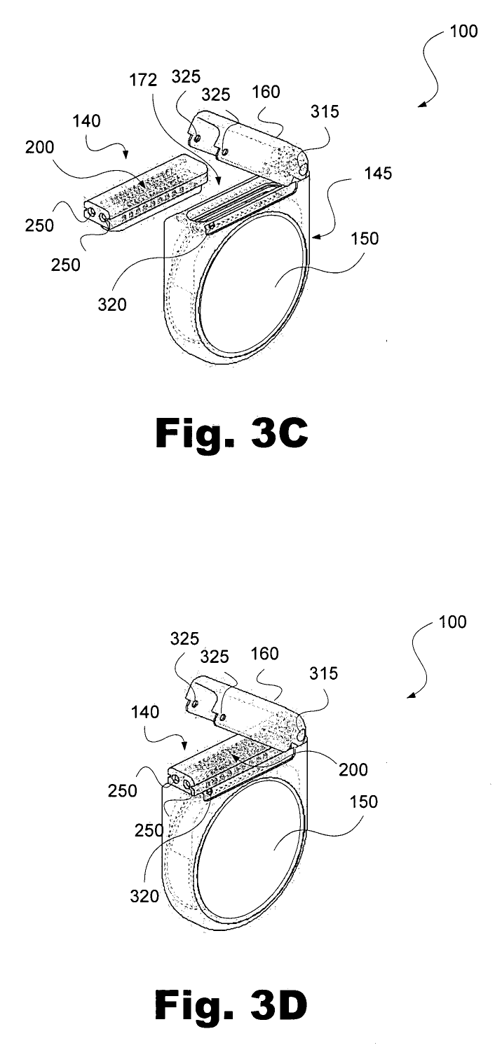

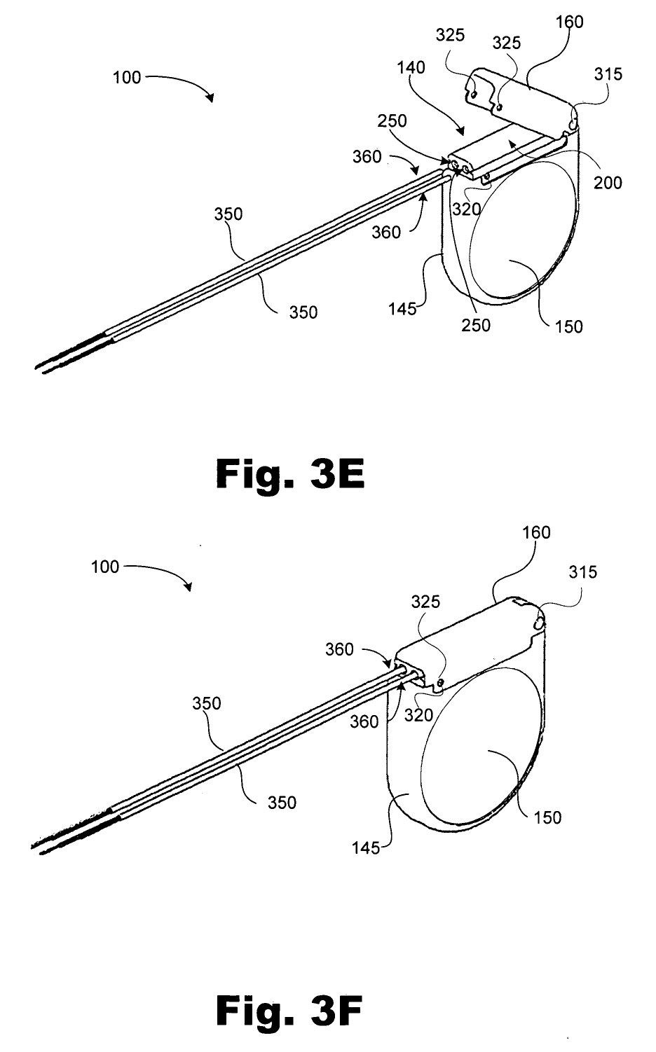

Further, the removable connector block (140) is configured to interact with the connector block cover (160). The connector block cover (160) may be shaped to fit over the connector block (140) and be secured to the case frame (145) using a locking mechanism. The interaction between the connector block cover (160) and the connector block (140) will be described in further detail below with reference to FIGS. 3A through 3F.

FIG. 2 illustrates an exploded view of a removable connector block (140), according to one exemplary embodiment. As shown in FIG. 2, the connector block (140) generally includes a resilient biocompatible body (200) made from a compressible material, feedthru receptacles (210), and connector contacts (220). The feedthru receptacles (210) are electrically coupled to respective connector contacts (220). As previously discussed, the connector block (140) is configured to couple and secure an IPG (100; FIG. 1) to the proximal connector end of a stimulating lead or the male end of an extension lead as a result of a compressive force applied to the resilient body (200). Each of the components of the connector block (140) and their interaction, along with an exemplary case assembly will be discussed below.

The resilient body (200) of the connector block (140) may be made of any suitable material, such as a resilient biocompatible material. An exemplary resilient biocompatible material includes, without limitation, soft silicone. According to one exemplary embodiment, the resilient body (200) is made of a resilient biocompatible material that is substantially transparent. Forming the resilient body (200) with a substantially optically transparent material allows a user to visually confirm correct lead insertion.

As illustrated in FIG. 2, the resilient body (200) of the connector block (140) generally includes a feedthru portion (230) and a lead insertion portion (240). The feedthru portion (230) of the resilient body (200) is configured to interact with and form an electrical connection with feedthru member (155). More specifically, as illustrated in FIG. 2, the feedthru portion (230) of the resilient body (200) includes a plurality of feedthru receptacles (210) arranged therein. According to the exemplary embodiment, the feedthru receptacles (210) are spaced within the feedthru portion (230) of the resilient body (200) perpendicular to the lead insertion portion (240) of the resilient body.

According to the exemplary embodiment illustrated in FIG. 2, the feedthru receptacles (210) are generally arcuate or ring-shaped contacts configured to electrically couple feedthru pins (170). The feedthru receptacles (210) may be made of any suitable biocompatible metallic material including, but in no way limited to, platinum and platinum/iridium. As previously discussed, the feedthru receptacles (210) are configured to be coupled to feedthru pins (170) formed on the feedthru member (155). Consequently, the feedthru receptacles (210) are positioned in an array that substantially corresponds with, and may mate with, the feedthru pins (170) of the feedthru member (155).

Additionally, the resilient body (200) includes a lead insertion portion (240) configured to receive a lead or lead extension. As shown in FIG. 2, the lead insertion portion (240) includes at least one lead insertion lumen (250) having a number of connector contacts (220) disposed therein. The lumen (250) defined by the resilient body (200) extends from a first end of the resilient body to a second end thereof and is configured to receive the proximal, connector end of a stimulation lead or lead extension without the application of insertion force. Further, as illustrated in FIG. 2, the connector contacts (220) are disposed along the length of the lumen (250).

In particular, the connector contacts (220) may be positioned at regularly spaced intervals within the lumen, such that upon insertion of a stimulation lead or lead extension, the connector contacts may be coupled to the lead or lead extension. The connector contacts (220) may be positioned so as to be in physical contact with associated feedthru receptacles (210). Consequently, the feedthru receptacles (210) are electrically coupled to the connector contacts (220). The connector contacts (220), according to the present exemplary embodiment, are generally arcuate or ring-shaped connector block contacts having a gap therein. The connector contacts (220) may be made of any suitable biocompatible metallic material. Exemplary biocompatible metallic materials include, without limitation, platinum and platinum/iridium.

FIGS. 3A and 3B illustrate one exemplary locking mechanism that may be used to facilitate compression of the connector block (140) during use. As illustrated in FIG. 3A, the case frame (145) includes a cavity (165) configured to house a circuit board (110; FIG. 1) and a feedthru opening (172) configured to seat a feedthru member (155; FIG. 1). Additionally, the case frame (145) includes a pivot point (300). A pin orifice (310) configured to receive a hinge pin (not shown) is also formed in the pivot point (300). FIG. 3A further illustrates a locking protrusion (320) formed on one side of the case frame (145). Another locking protrusion (not shown) is placed on the opposite side of the case frame. According to one exemplary embodiment illustrated below, the locking protrusions (320) are configured to interact with and securely lock the connector block cover (160; FIG. 1) to the case frame (145).

As illustrated in FIG. 3B, the connector block cover (160) also includes a pin orifice (312) formed in a first end of the connector block cover. According to the present exemplary embodiment, the pin orifice (312) of the connector block is configured to be concentrically aligned with the pin orifice (310; FIG. 3A) of the pivot protrusion (300; FIG. 3A) during assembly, thereby forming a lumen configured to allow the insertion of a hinge pin (not shown). Additionally, a pair of lock receiving orifices (325), for each receiving a locking protrusion (320; FIG. 3A), are formed in the connector block cover (160) opposite the pin orifice (312). According to the present exemplary embodiment, the lock receiving orifices (325) are configured to lockingly interact with the locking protrusion (320; FIG. 3A) of the case frame (145; FIG. 3A).

FIG. 3C illustrates an assembled case frame (145) and connector block cover (160), according to one exemplary embodiment. As illustrated in FIG. 3C, a hinge pin (315) is inserted into concentrically aligned pin orifices (310, 312; FIGS. 3A and 3B respectively) allowing the connector block cover (160) to pivot on a first end. Additionally, as illustrated in FIG. 3C, the insertion of the hinge pin (315) allows the lock receiving orifices (325) to be rotatably aligned with the locking protrusion. More specifically, the connector block cover (160) may rotate about the hinge pin (315) such that the lock receiving orifices (325) engage the locking protrusions (320) formed on the case frame (145).

FIG. 3C also illustrates the insertion of a removable connector block (140) into the assembly. According to the present exemplary embodiment, the hinging of the connector block cover (160) on the hinge pin (315) allows for the selective insertion and/or removal of the connector block (140). According to this exemplary embodiment, allowing the connector block (140) to be selectively removed from the IPG (100) facilitates the use of interchangeable and/or replacement connector blocks, as desired. The ability to interchange and/or replace the connector block allows for the incorporation of connector blocks with different insertion lumens configured to accommodate different lead connector sizes without varying the structure or configuration of the case. In contrast to traditional IPG connectors, the present exemplary embodiment allows a single IPG (100) to be connected with various connector blocks having any number of stimulation leads and having different proximal connector sizes and configurations (e.g. 4 contacts versus 8 contacts).

As illustrated in FIG. 3D, the connector block (140) may be seated in the feedthru opening (172; FIG. 3C) formed in the case frame (145). As the feedthru portion (230; FIG. 2) of the connector block (140) is seated in the feedthru opening (172), the feedthru receptacles (210; FIG. 2) are placed in electrical contact with corresponding feedthru pins (170; FIG. 2). The insertion of the connector block (140) establishes an electrical connection from the circuit board (110; FIG. 1), via the hybrid pins (130; FIG. 1), the flexible connector (135; FIG. 1), the feedthru member (155; FIG. 1), and the feedthru receptacles (210; FIG. 2), to the connector contacts (220; FIG. 2) of the connector block (140).

FIGS. 3E and 3F illustrate perspective views of the case frame (145), the connector block cover (160), the connector block (140), and proximal end (360) of two leads or lead extensions (350). In particular, FIG. 3E illustrates the connector block cover (160) open relative to the case frame (145) and the connector block (140), which connector block is fitted and attached to the case frame (145) as previously described. FIG. 3F illustrates the connector block cover (160) in a locked or closed position relative to the case frame (145) enclosing the connector block (140). When the connector block cover (160) is in the closed position and locked, the proximal end (360) of the lead extensions or lead (350) are securely coupled to the connector block (140). For purposes of definition herein, a lead extension has a proximal connector end, which is intended to be inserted into the lead insertion lumen of an IPG. The distal end of a lead extension conventionally has a female receptacle for accepting the connector end of a stimulation lead. The lead extensions or leads (350) may be made of any suitable biocompatible conductor material covered with an outer insulative material. Exemplary conductor materials include, without limitation, wire material such as MP35, platinum/iridium alloy, platininum wire, or other suitable conductor material. The proximal ends (360) of the lead extensions or leads (350) are configured to be coupled to the connector block (140).

The connector contacts (220; FIG. 2) are generally ring shaped with a gap formed therein breaking up the ring. The connector contacts are lined up in an array formation and located within the lead insertion lumens or channels (250) formed in the resilient body (200). While the connector block cover (160) is open relative to the case frame (145) as shown in FIG. 3E, the resilient body (200) and the connector contacts (220; FIG. 2) are uncompressed. While the resilient body (200) and the connector contacts (220) are thus uncompressed, the proximal ends (360) of the lead extensions or stimulation leads (350) may be placed within the lumens (250) without obstruction or essentially at zero insertion force. Zero insertion force is achieved because the diameter of the lead insertion lumen (250) is greater than the diameter of the proximal end of the stimulating lead or lead extension that is designed to be inserted into the lead insertion lumen (250). This ability to insert the lead at essentially zero insertion force is a major advantage over conventional lead connection systems which generally require some insertion force. When an insertion force is needed to insert a lead, the practitioner will sometimes guess where the end of the insertion lumen is. Other times, the insertion forces may cause the proximal end (360) of the lead or extension lead to jam inside the lumen and not insert fully into the lumen. These situations can cause the lead or extension lead to become damaged, causing undesirable scrapping of the lead, as well as passage of precious surgical operating room (O.R.) time. With zero insertion force, as provided with one embodiment of the present connector system, placing the leads into the insertion lumens within the connector block during a surgical implantation procedure is much less cumbersome and less time consuming. A practitioner can insert the proximal end of the lead or lead extension into the lumen, through the connector contacts (220; FIG. 2) until the lead or lead extension positively abuts the end of the lumen (250).

After the proximal ends (360) of the lead extensions or leads (350) are passed through the connector contacts (220; FIG. 2), the connector cover (160) may be secured in the closed position to the case frame (145) by employing the locking mechanism. According to the exemplary embodiment illustrated in FIG. 3F, the locking mechanism includes pivoting the connector cover (160) about the hinge pin (315) until the lock receiving orifices (325) formed in the connector cover overlap and engage the locking protrusions (320) formed in the case frame (145). As the connector block cover (160) is closed and secured to the case frame (145), the connector block cover (160) and case frame (145) exert compressive forces on the resilient body (200). The compressive forces produced by the connector block cover (160) and the case frame (145) are then transferred to the connector contacts (220). As previously discussed, the connector contacts (220) have gaps therein. The compressive forces compress the connector contacts (220), thereby causing the gaps to narrow or close. As the gaps narrow, the connector contacts (220) transfer the compressive forces to the proximal, connector end of a lead or lead extension (350), such that the connector contacts (220) are clamped to the proximal connector end, forming an electrical connection. Further, the compressive forces applied to the connector block (140) as the connector block cover (160) is closed relative to the case frame (145) causes the feedthru portion (230) of the connector block to be compressed and securely couple the feedthru pins (170; FIG. 2) of the feedthru member (155; FIG. 2).

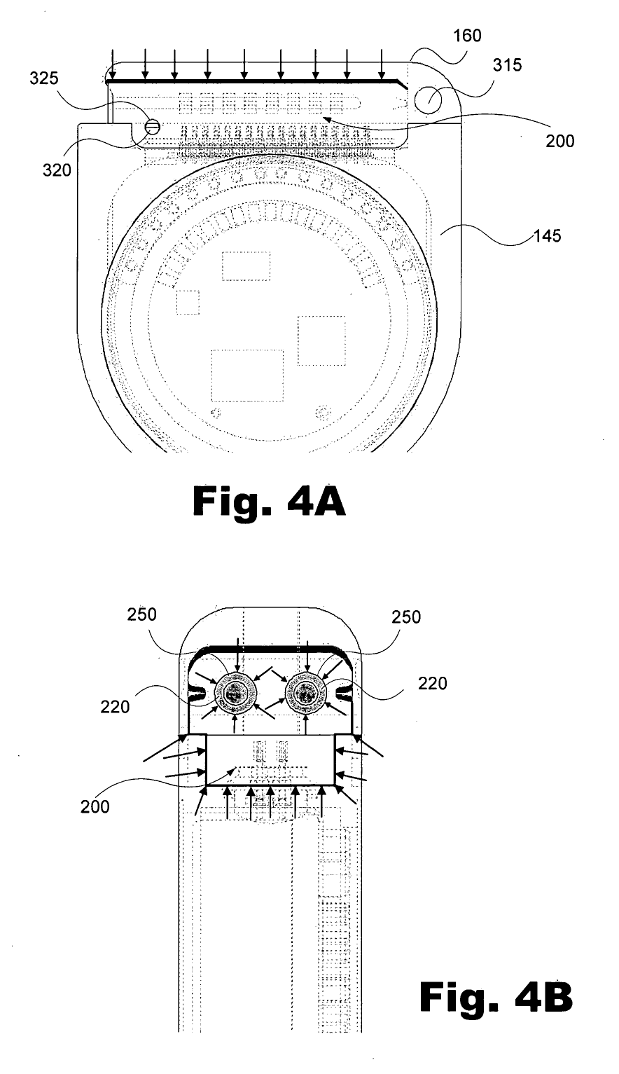

In such a configuration, when the connector block cover (160) is closed relative to the case frame (145), the connector block cover (160) exerts a compressive force on the connector block (140) as illustrated in FIGS. 4A and 4B. As illustrated by the force arrows in FIG. 4A, pivoting the connector block cover (160) with respect to the case frame (145), such that the lock receiving orifice (325) engages the locking protrusion (320), generates a compressive force on the resilient body (200). As mentioned previously, the compressive force exerted on the resilient body (200) is transferred to the connector contacts (220; FIG. 2) allowing a lead or lead extension to be securely coupled thereto.

In some embodiments of the connector block, which are within the scope of the present invention, the connector block (160) is formed of a non-compressible material that uses a conventional, friction-fit lead insertion lumen and connector contacts. However, the removable connector block may be attached and removed from the case frame, by utilizing a movable connector block cover (160) to secure the connector block to the case frame. One embodiment of the connector block (140) does not offer a zero insertion force into the insertion lumen, although the particular embodiment of the connector block does offer selective attachment or detachment of the connector block to the case frame.

In another embodiment, the connector block may be permanently attached to the case frame of a medical device. However, the connector block may be made from resiliently compressible material and have within the connector block a connector contact or connector contacts that respond and conform to compressive forces exerted on the resilient connector block and thereby securely clamp down on the inserted proximal connector end of a lead or lead extension.

FIG. 4B further illustrates the compressive force exerted by locking the connector block cover (160; FIG. 4A). As illustrated in FIG. 4B, the compressive forces act radially inward on the lead insertion lumens (250), thereby compressing the connector contacts (220). The compression of the connector contacts (220) results in a narrowing or closing of the gaps formed therein. As the gaps narrow or close, the connector contacts (220) transfer the compressive forces to the lead extensions (350), such that the connector contacts (220) are clamped to the stimulating lead or lead extensions, forming an electrical connection. Accordingly, the connector (140) is configured to be simultaneously coupled to a lead extension and the feedthru member (155) as the connector block cover (160) exerts a compressive force on the connector (140). Additionally, the compressive force reduces the diameter of the lumen (250) such that the material comprising the resilient body (200) frictionally resists extraction of the lead or lead extension (350) when the connector block cover (160; FIG. 4A) is locked to the case frame (145).

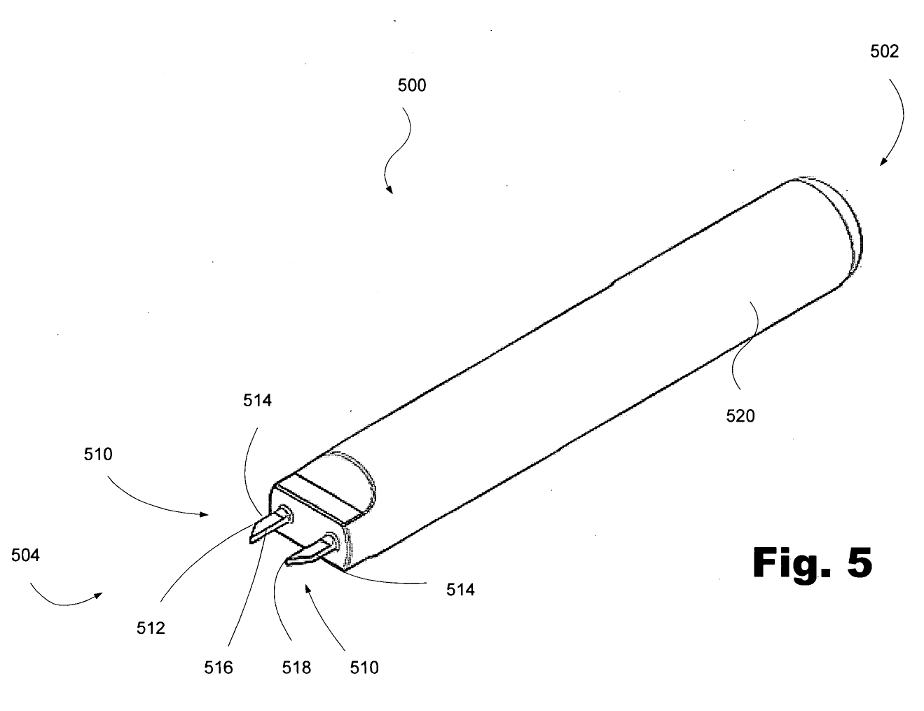

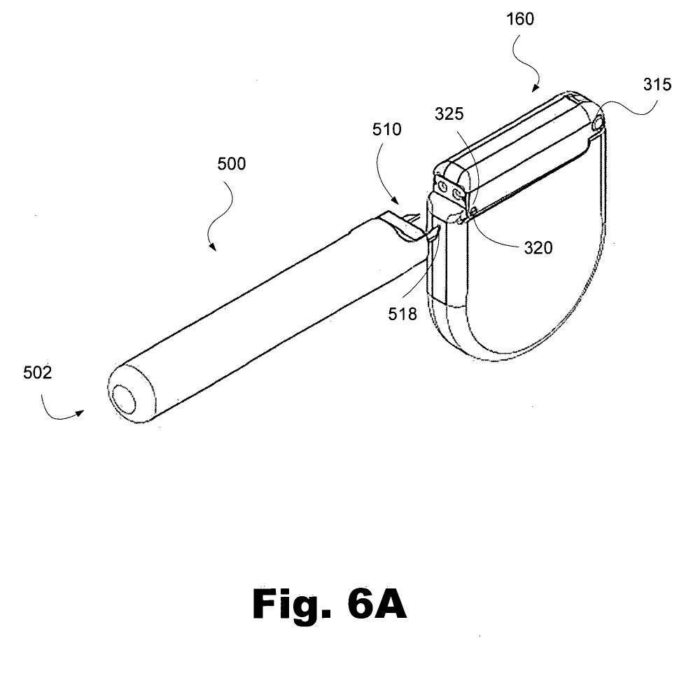

Under some circumstances, it may be desired to open or unlock the connector block cover (160) relative to the case frame (145) after it has been secured. For example, if the proximal end (360; FIGS. 3E and 3F) of the lead is not inserted fully into the insertion lumen (250), the lead may need to be re-inserted. Hence, the connector block cover (160) must be unlocked. To unlock the exemplary connector block cover (160), the walls of the connector block cover (160) having the lock receiving orifice (325) formed therein may be spread apart to eliminate the interference between the lock receiving orifices (325) and the locking protrusion (320). According to one exemplary embodiment, the prying tool (500) illustrated in FIG. 5 may be used to spread the walls of the connector block cover (160) sufficiently to unlock the connector block cover. As illustrated in FIG. 5, a prying tool (500) includes a body portion (520) having a first (502) and a second (504) end. The body portion (520) is configured to serve as a handle for the operation of the prying tool (500).

Additionally, as illustrated in FIG. 5, the second end (504) of the prying tool (500) includes a number of prongs (510) projecting therefrom. According to the illustrated embodiment, the prongs each include an external cover edge (514) and an internal lock edge (516) formed substantially parallel with the longitudinal axis of the prying tool (500). A first end of each prong terminates with an inclined face (512) forming a point (518) with the lock edge (516) as shown.

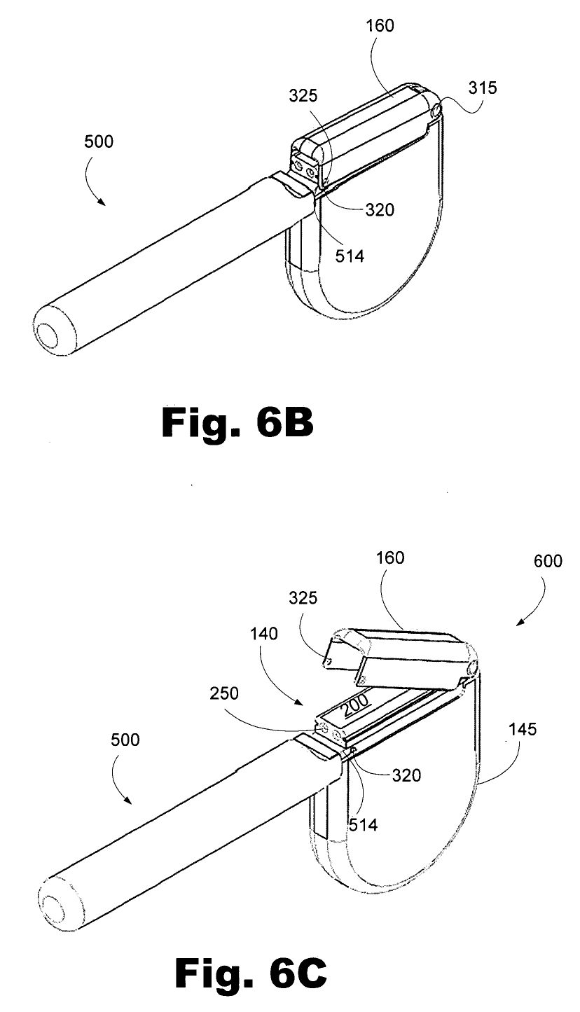

FIGS. 6A through 6C illustrate an exemplary method for opening or unlocking a connector block cover (160) relative to the case frame (145) after it has been secured, using the present prying tool (500). As illustrated in FIG. 6A, the second end of the prying tool (500) having the prying prongs (510) formed thereon is presented adjacent to the connector block cover (160) where the lock receiving orifices (325) engage the locking protrusion (320).

The prying prongs (510) of the prying tool (500) are then inserted between the connector block cover (160) and the case frame (145) as illustrated in FIG. 6B. According to the present exemplary embodiment, the point (518) of each prying prong (510) initiates the insertion and the lock edge (516) of the prong then follows the profile of the case frame (145). As the lock edge (516) of each prying prong (510) follows the profile of the case frame (145), the inclined faces (512) force the walls of the connector block cover (160) away from the case frame (145). As the walls of the connector block cover (160) are forced away from the case frame (145), the lock receiving orifices (325) are also forced away from the locking protrusions (320), thereby eliminating the interference between them.

As illustrated in FIG. 6C, with the interference between the lock receiving orifice (325) and the locking protrusion (320) eliminated, the connector block cover (160) is unlocked and may be opened. When the case cover (160) is thereby opened, the compressive forces to the connector block (140) and connector contacts (220; FIG. 2) are not applied. As these compressive forces are removed the resilient body (200) and the connector contacts (220; FIG. 2) substantially return to their uncompressed shapes. Consequently the lead or lead extensions (350) may thereby be removed from connector block (140) or reinserted into the insertion lumen in a zero insertion force environment.

FIG. 6C illustrates an example embodiment of a complete implantable stimulator system (600), in accordance with the invention. According to the exemplary embodiment illustrated in FIG. 6C, the implantable stimulator system may include a medical device, having a case and a case frame (145), a removable connector block (140) having a lead insertion lumen (250), which lumen is dimensioned to a size larger than a connector end of a stimulation lead or a lead extension. Additionally, a pivotable connector block cover (160) is attached on one end of the housing to the implantable medical device. As illustrated in FIGS. 6A through 6C, the connector block cover (160) has an open, unlocked position and a locked, closed position. As previously described, when in the locked position, the removable connector block (140) is securely attached to the medical device case. An opening or prying tool (500) also forms part of the stimulator system (600) illustrated in FIG. 6C. As explained previously, the opening tool is configured to open the connector block cover (160) and release the connector block (140) from the medical device case.

A method of using the above-mentioned stimulator system includes, but is in no way limited to, providing a stimulator case configured to accept a lead connector block, selecting a stimulation lead or extension lead with a proximal connector end having a predetermined size, selecting a removable lead connector block with a lead insertion lumen sized to accept the proximal connector end of the stimulation lead or extension lead, securing the lead connector block to the stimulator, securely inserting the proximal connector end of the stimulation lead or extension lead into the insertion lumen, if an extension lead has been inserted into the insertion lumen, attaching the, proximal connector end of the selected stimulation lead to the distal female of the extension lead, and implanting the stimulator and connected stimulation lead into a patient.

The stimulator device or IPG (100), according to the present exemplary embodiment, includes a case frame (145) with side lids (150) described therein. Those of skill in the art will appreciate that any type of case may be used with a removable connector. Further, the feedthru receptacles (210) and connector contacts (220) shown and described with reference to the present exemplary embodiment may be ring-shaped with gaps defined therein. Those of skill in the art will appreciate that feedthru receptacles and connector contacts of any shape or configuration may be used in a connector. Moreover, while the present locking mechanism, configured to maintain compression on the connector block (140) from the case cover (160), is described in the context of a locking protrusion and receiving orifice or hole interference, any number of locking mechanisms may be used to maintain the desired pressure on the connector block (140), as will be readily appreciated by one of ordinary skill in the art.

In conclusion, a connector is provided herein for use in a stimulator device. In particular, the connector may include a resilient body having a lumen defined therein. Connector contacts are disposed along the length of the lumen. The connector contacts are configured to be coupled to lead extensions or leads, which direct electrical stimulation to a desired body location. As described herein, the connector may include a zero-insertion force configuration while maintaining electrical contact without the use of a set screw. According to one exemplary embodiment, the resilient body is relatively clear so that the male end of an extension lead or the proximal connector end of a stimulating lead can be seen as it is inserted into the insertion lumen within the removable connector block to facilitate a correct insertion. Further, the present connector is easily modified and/or replaced with a connector block having a different configuration and/or different lumen size.

When the connector block is subjected to compressive forces, the compressive forces are transferred to the lead insertion lumen and the connector contacts in the connector block. When a lead or lead extension is placed at least partially within the lumens such that part of the lead or lead extension is also passed through the connector contacts, the compressive forces securely clamp the connector contacts to the lead extensions. Because the size of the lead insertion lumen also decreases as a result of applied compressive forces, particularly at the lumen surface between the connector contacts, a fluid seal is formed between the lead insertion lumen and proximal connector end of an inserted lead or lead extension. Further, the connector contacts are also coupled to feedthru receptacles, which in turn are coupled to a feedthru member, which is electrically coupled to the circuit board.

The preceding description has been presented only to illustrate and describe the present method and apparatus. It is not intended to be exhaustive or to limit the disclosure to any precise form disclosed. Many modifications and variations are possible in light of the above teaching. It is intended that the scope of the disclosure be defined by the following claims.

What is claimed is:

1. A connector block for use in an implantable stimulator device, comprising:
a resilient, substantially compressible body having at least one lead insertion lumen defined in said resilient body; and
a plurality of connector contacts disposed within said lumen, said contacts being configured to yield to compressive forces in response to external compressive forces applied to the resilient, substantially compressible body and thereby exert a clamping force on the connector end of a lead or a lead extension.

2. The connector block of claim 1, wherein said at least one insertion lumen is dimensioned to be larger than the connector end of a lead or a lead extension for zero insertion force.

3. The connector block of claim 1, wherein said resilient body comprises silicone.

4. The connector block of claim 1, wherein said resilient body comprises a substantially transparent material.

5. The connector block of claim 1, wherein said connector block is configured to permit selective attachment and detachment from a case frame of the implantable stimulator.

6. The connector block of claim 5, further comprising:
a plurality of feedthru receptacles coupled to said connector contacts, said feedthru receptacles being configured to be electrically coupled to a feedthru member.

7. The connector block of claim 1, wherein said connector contacts comprise a substantially ring-shaped body with a gap formed in said ring-shaped body.

8. The connector block of claim 7, wherein said connector contacts comprise a biocompatible metallic material.

9. The connector block of claim 8, wherein said biocompatible metallic material comprises one of platinum or platinum/iridium alloy.

10. An implantable stimulator device, comprising:
a circuit board;
a case assembly having a case frame; and
a removable, lead connector block having at least one lead insertion lumen defined in said connector block and a plurality of connector contacts disposed within said lumen, wherein the lead connector block is configured to be attachable to the case frame.

11. The implantable stimulator device of claim 10, wherein the removable lead connector block and case frame are each configured to permit selective attachment and detachment of the lead connector block to the case frame.

12. The implantable stimulator device of claim 10, further comprising:
a connector block cover that is dimensioned to fit over the lead connector block, wherein the connector block cover is pivotably attached to the case frame, the connecter block cover having a locked and unlocked position, wherein in the locked position, the lead connector block is secured to the case frame.

13. The device of claim 12, wherein said case frame comprises:
a locking protrusion; and
a connector block cover having a lock receiving orifice for accepting the locking protrusion, wherein said connector block cover, lead connector block, and case frame are configured such that, when said connector block cover is secured in the locked position, and said lock receiving orifice engages with said locking protrusion, a compressive force is applied to said lead connector block causing the lead insertion lumen to decrease in size.

14. An implantable stimulator system comprising:
a medical device, having a case;
a removable connector block having a lead insertion lumen, which lumen is dimensioned to a size larger than a connector end of a stimulation lead or a lead extension;
a pivotable connector block cover, attached on end of the case to the implantable medical device, the connector block cover having an open, unlocked position and a locked, closed position, wherein in the locked position, the removable connector block is securely attached to the medical device case; and
an opening tool, configured to open the connector block cover and release the connector block from the medical device case.

15. The stimulator system of claim 14, wherein the medical device case has a locking protrusion and the connector block cover has a complementary lock receiving orifice for accepting the locking protrusion into the lock receiving orifice in the locked position; and
wherein the opening tool is a prying tool configured to separate the connector block cover from the medical device case to permit the connector block cover to be placed into the unlocked position.

16. The stimulator system of claim 15, wherein the opening tool has at least one prong, which prong is used to pry apart the connector block cover from the medical device case.

17. The stimulator system of claim 15, further comprising:
a plurality of removable connector blocks, each connector block having an insertion lumen dimensioned to a size for accepting a different sized connector end of a lead or lead extension.

18. A method of using a stimulator system comprising:
providing a stimulator device case configured to accept a removable lead connector block;
selecting a stimulation lead or extension lead with a proximal connector end having a predetermined size;
selecting a removable lead connector block with a lead insertion lumen sized to accept the proximal connector end of the stimulation lead or extension lead;
securely inserting the proximal connector end of the stimulation lead or extension lead into the insertion lumen;
securing the lead connector block to the stimulator;
if an extension lead has been inserted into the insertion lumen, attaching the, proximal connector end of the selected stimulation lead to the distal female of the extension lead; and
implanting the stimulator and connected stimulation lead into a patient.

19. The method of using a stimulator system of claim 18, further comprising:
releasing the removable lead connector block from the stimulator.

20. The method of using a stimulator system of claim 19, further comprising:
releasing the connected proximal end of the lead or lead extension from the removable lead connector block.

21. The method of using a stimulator system of claim 18, wherein securing the lead connector block to the stimulator comprises compressing the lead connector block and compressing the lead insertion lumen over the inserted proximal connector end of the stimulation lead or extension lead.

22. The method of using a stimulator system of claim 21, wherein compressing the lead connector block and compressing the lead insertion lumen comprises placing a pivotable connector block cover into a locked position, wherein the connector block cover has a locked position and unlocked position.
